(19) United States
(12) Patent Application Publication
(10) Pub. No.: US 2004/0126782 A1
(43) Pub. Date: Jul. 1, 2004

(54) System and method for SNP genotype clustering

(76) Inventors: David P. Holden, Burlingame, CA (US); XiaoPing Zhang, Mississauga, CA (US); Daniel B. Allison, Indianapolis, IN (US); Austin B. Tomaney, San Francisco, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/611,414

(22) Filed: Jun. 30, 2003

Related U.S. Application Data
(60) Provisional application No. 60/392,841, filed on Jun. 28, 2002.

(51) Int. Cl. C12Q 1/68; G06F 19/00; G01N 33/48; G01N 33/50

(52) U.S. Cl. 435/6; 702/20

(57) ABSTRACT

A system and methods for evaluating genetic information and biological data applying a clustering approach which may be used for allele calling and genotyping. Statistical analysis of sample data is performed at various levels to develop a model which associates individual data points with selected genotyping clusters and provides a relative indication of the call confidence. The methods provide a unified framework for allele-calling in many different contexts and may be applied to the data acquired from various identification methodologies.

### CLAIM OF PRIORITY

This U.S. patent application claims priority to U.S. Provisional Patent Application No. 60/392841 entitled “A method for SNP Genotype Clustering Using Error Weighted Seed Clustering” filed Jun. 28, 2002 which is hereby incorporated by reference and U.S. Provisional Patent Application filed Jun. 30, 2003, entitled “System and Method for SNP Algorithm and Data Validation” (Atty Docket No. ABIOS.056PR) which is hereby incorporated by reference.

### BACKGROUND

1. Field

The present teachings generally relate to the field of genetic analysis and more particularly to a system and methods for analysis of biological information using a data clustering approach.

2. Description of the Related Art

Cluster analysis is an analytical paradigm frequently used to identify correlations and patterns in data. In the context of biological and genetic research, clustering approaches may be used for the purposes of allelic classification and analysis of genetic sequence variations including insertions, deletions, restriction fragment length polymorphisms (“RFLPs”), short tandem repeat polymorphisms (“STRPs”), and single nucleotide polymorphisms (“SNPs”). In general, clustering approaches attempt to classify a data point by relating it to other data points from a selected sample set. For example, in an exemplary SNP analysis, fluorescent probes may be used in the generation of amplification products for a large number of samples. The fluorescence values for each sample are quantitated and then classified with respect to one another by plotting the fluorescence values of the entire set on a two dimensional graph or scatterplot. When plotted in this manner it may be observed that the data tends to aggregate into discrete groupings according to geneotype. Using this information, a human observer may be able to distinguish the various groupings or clusters of data and classify individual data points according to the cluster in which they reside to determine the geneotype for a selected sample.

One significant limitation which impedes many conventional methods for clustering analysis of biological data is that it becomes increasingly time consuming and laborious to perform an analysis as the size of the sample set increases. This problem is exacerbated when experimental data points cannot be readily associated with a single cluster and as a consequence the development of automated clustering tools may be significantly hindered due to the inability of these tools to resolve such data points. In order to overcome these limitations it is desirable to develop a rapid, reliable, and unsupervised method for computational analysis that is capable of a level of throughput necessary to analyze large sample sets. Furthermore, it is desirable to provide an analytical approach that is able to classify data points whose characteristics are ambiguous or difficult characterize with respect to other data points in the sample set.

### SUMMARY

In various embodiments the present teachings describe a system and methods for performing allelic classification and genotyping by developing a statistical model based for cluster-based analysis in which error information for each data point is used to determine a statistically valid cluster or class to which it belongs. The statistical model implements a composite analysis which can be decomposed into probabilities associated with the model itself, the individual data points, and the clusters formed by the data points. In general, the allelic classification methods may operate in an unsupervised manner (e.g. no requisite training data necessary) with relatively little knowledge required about the sample set aside from the raw input values.

In one aspect, the present teachings describe a method for allelic classification, the method comprising: (a) acquiring intensity information for a plurality of samples wherein the intensity information comprises a first intensity component associated with a first allele and a second intensity component associated with a second allele; (b) evaluating the intensity information for each of the plurality of samples to identify one or more data clusters, each cluster associated with a discrete allelic combination and determined, in part, by comparing the first intensity component relative to the second intensity component; (c) generating a likelihood model that predicts the probability that a selected sample will reside within a particular data cluster based upon its intensity information; and (d) applying the likelihood model to each of the plurality of samples to determine its associated allelic composition.

In another aspect, the present teachings describe a method for clustering analysis, the method comprising: (a) identifying a sample set comprising a plurality of data points, each data point having an angular value representative of an association between a first and a second intensity component; (b) generating a likelihood model and associated parameter set wherein the angular values of the data points are used in determining the appropriate parameters to be used in the likelihood model and wherein the efficacy of the likelihood model is assessed by evaluating the probability the likelihood model properly identifies selected data points in the sample set; (c) applying the likelihood model to the plurality of data points within the sample set and grouping the data points into discrete clusters; and (d) associating a selected classification with each discrete cluster and its component data points.

In still another aspect, the present teachings describe a method for allelic classification, the method comprising: (a) identifying a sample set comprising a plurality of data points each having at least two component intensity values; (b) evaluating the component intensity values for the plurality of data points to group the data points into one or more data clusters representative of discrete allelic classifications; (c) generating a likelihood function that describes the grouping of a selected data point using its component intensity value; and (d) associating an allelic classification with each data point using the likelihood function.

In another embodiment, the present teachings describe a computer readable medium having stored thereon instructions which cause a general purpose computer to perform the steps of: (a) acquiring experimental information for a plurality of samples wherein the experimental information comprises a first data component associated with a first allele and a second data component associated with a second allele; (b) evaluating the experimental information for each of the plurality of samples to identify one or more data clusters, each cluster associated with a discrete allelic combination and determined, in part, by comparing the first, data component relative to the second data component; (c) generating a likelihood model that predicts the probability that a selected sample will reside within a particular data cluster based upon its experimental information; and (d) applying the likelihood model to each of the plurality of samples to determine its associated allelic composition.

In still another embodiment, the present teachings describe a computer readable medium having stored thereon instructions which cause a general purpose computer to perform the steps of: (a) identifying a sample set comprising a plurality of data points, each data point having an angular value representative of an association between a first and a second intensity component; (b) generating a likelihood model and associated parameter set wherein the angular values of the data points are used in determining the appropriate parameters to be used in the likelihood model and wherein the efficacy of the likelihood model is assessed by evaluating the probability the likelihood model properly identifies selected data points in the sample set; (c) applying the likelihood model to the plurality of data points within the sample set and grouping the data points into discrete clusters; and (d) associating a selected classification with each discrete cluster and its component data points.

In another aspect, the present teachings describe a computer readable medium having stored thereon instructions which cause a general purpose computer to perform the steps of: (a) identifying a sample set comprising a plurality of data points each having at least two component experimental values; (b) evaluating the component experimental values for the plurality of data points to group the data points into one or more data clusters representative of discrete allelic classifications; (c) generating a likelihood function that describes the grouping of a selected data point using its component experimental value; and (d) associating an allelic classification with each data point using the likelihood function.

In still another aspect, the present teachings describe a computer-based system for performing allelic classification, the system comprising: a database for storing experimental information for a plurality of samples, the experimental information reflecting the allelic composition of each sample and a program which performs the operations of: (a) retrieving experimental information for the plurality of samples from the database wherein the experimental information comprises a first data component associated with a first allele and a second data component associated with a second allele; (b) evaluating the experimental information for each of the plurality of samples to identify one or more data clusters, each cluster associated with a discrete allelic combination and determined, in part, by comparing the first experimental component relative to the experimental component; (c) generating a likelihood model comprising a model-fit probability assessment that estimates confidence in the likelihood model itself and assesses how well a selected sample and its respective experimental information fit the model, the model further used to predict the probability that a selected sample is associated with a particular data cluster based upon its experimental information; and (d) applying the likelihood model to each of the plurality of samples to determine its associated allelic composition.

In another embodiment, the present teachings describe a computer-based system for performing allelic classification, the system comprising: a database for storing experimental information for a plurality of samples, the experimental information reflecting the allelic composition of each sample; and a program which performs the operations of: (a) identifying a sample set comprising a plurality of data points, each data point having an angular value representative of an association between a first and a second intensity component; (b) generating a likelihood model and associated parameter set wherein the angular values of the data points are used in determining the appropriate parameters to be used in the likelihood model and wherein the efficacy of the likelihood model is assessed by evaluating the probability the likelihood model properly identifies selected data points in the sample set; (c) applying the likelihood model to the plurality of data points within the sample set and grouping the data points into discrete clusters; and (d) associating a selected classification with each discrete cluster and its component data points.

### BRIEF DESCRIPTION OF THE DRAWINGS

FIG. 1A is a scatterplot of raw fluorescence intensity data acquired for a plurality of data points.

FIG. 1B is an exemplary sample set in which fluorescence intensity data is plotted as a log function scatterplot.

FIG. 1C is a scatterplot in which each cluster or allelic grouping is associated with a discrete angular value.

FIG. 1D is an exemplary polar plot for intensity values for a plurality of data point plotted as a function of angle values.

FIG. 2 is a generalized method for single nucleotide polymorphism analysis.

FIG. 3 is a method for data classification incorporating a maximum likelihood analytical approach.

FIG. 4 is a block diagram illustrating the components of a combined probability analysis for data classification.

FIG. 5 is an exemplary angle space Gaussian function used in clustering analysis.

FIG. 6 is a method for array-based analysis incorporating the maximum likelihood analytical approach.

FIG. 7 is an exemplary system for performing allelic classification.

### DETAILED DESCRIPTION OF CERTAIN EMBODIMENTS

The present teachings describe a clustering approach that may be used to evaluate genetic information and biological data. In one aspect, these methods may be adapted to a computerized analysis platform or software application wherein the data analysis is performed in a substantially automated manner. By providing a mechanism for automated data analysis, the present teachings effectively address many of the limitations of conventional methods which generally necessitate a human observer to evaluate individual data points. Furthermore, the methods described herein may improve the speed and accuracy of analysis for large sample sets to thereby improve the efficiency of analysis in high throughput applications.

In various embodiments, the present teachings may also be used to evaluate sample sets containing ambiguous or difficult to classify data points. This feature is particularly useful to classify data points that fall outside or on the boundaries of one or more clusters. Ambiguous data points present a significant problem in conventional clustering approaches as their classification is subject to an increased likelihood of “miscalling” resulting in improper identification or an erroneous association of the data point with a cluster to which it does not actually belong.

In certain embodiments, the present teachings may be adapted to operate in conjunction with a variety of different biological and genetic data analysis applications wherein clustering analysis is employed to resolve relationships between a plurality of data points which form a sample set. One exemplary application where clustering analysis may be used is in connection with locating or identifying SNPs and sample genotyping.

SNPs represent one of several types of nucleotide sequence variations that naturally occur and it is generally believed that detailed SNP analysis may be useful in studying the relationship between nucleotide sequence variations and diseases or other conditions. Currently, there are over 3 million putative SNPs that have been identified in the human genome and it is a goal of many researchers to verify these putative SNPs and associate them with phenotypes and diseases. One challenge in meeting this goal is that it is necessary for researchers to generate and analyze large amounts of genotypic data which in many instances may require careful investigator review and interpretation.

A number of analytical methods have been developed which can locate or identify SNPs. One exemplary method involves sample amplification using pairs of fluorescent probes wherein each probe comprises a discrete marker or reporter dye specific for a different allele. During amplification the sample is labeled according to its particular allelic composition and the fluorescent properties of the resulting product can be evaluated to determine if the sample is homozygous for a first allele (e.g. A/A), homozygous for a second allele (e.g. A/B), or a heterozygous allelic combination (e.g. B/B). Homozygous samples tend to exhibit an increased degree of fluorescence in one or the other marker type with the amount of observed fluorescence from the opposing marker being significantly diminished or completely absent. Conversely, a sample heterozygous for both alleles typically exhibits a substantial degree of fluorescence arising from both markers. A commercial implementation of this method is Applied Biosystems' Taqman platform, which employs Applied Biosystems' Prism 7700 and 7900HT sequence detection systems to monitor and record the fluorescence of each amplified sample.

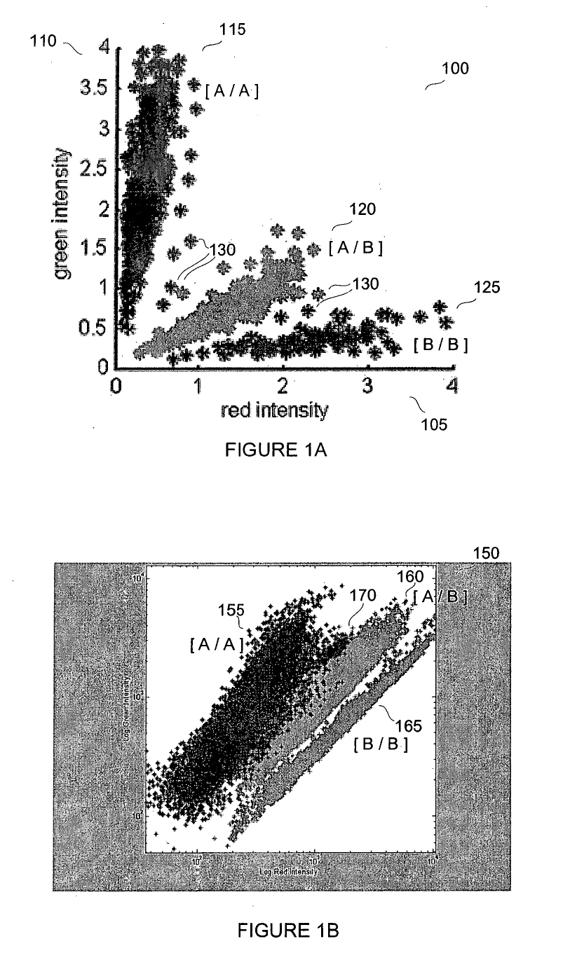

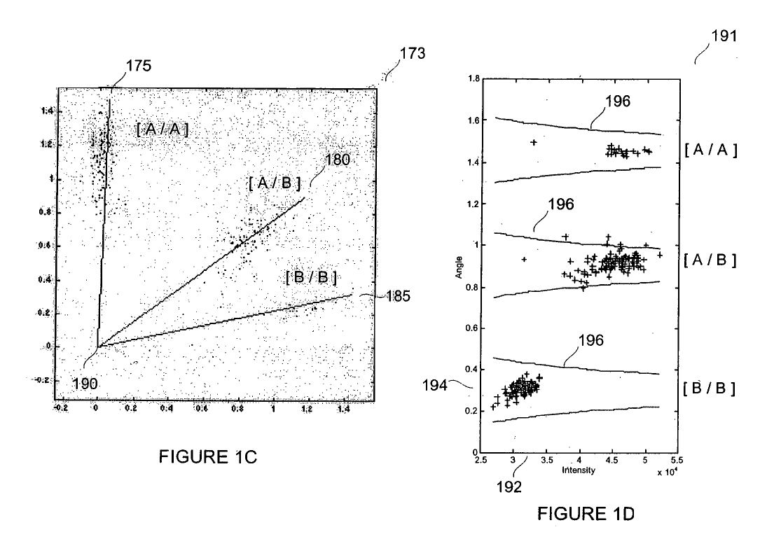

FIGS. 1A-D illustrate exemplary sample sets which might be acquired according to the aforementioned principals wherein fluorescence data from the amplification products for a plurality of samples is evaluated with respect to one another. In FIG. 1A, a scatterplot 100 may be used to visualize raw fluorescence intensity data acquired for a plurality of data points. In this representation 100, the x-axis 105 is associated with the fluorescence intensity associated with a first marker (red intensity) and the y-axis 110 represents fluorescence intensity for a second marker (green intensity). Thus each data point may be plotted with respect to other data points based on the measured fluorescence intensity values.

Allelic classification of individual samples within the sample set may be accomplished by evaluating the measured fluorescence values for the entire sample set with respect to on another. Visualization of the exemplary data via the scatterplot 100 indicates that the data points tend to cluster into separate groupings 115, 120, 125. These groupings 115, 120, 125 may further be associated with a particular allelic composition or geneotype as shown wherein the first group 115, represents those samples having a homozygous allelic composition of [A/A]. The second group 120, represents those samples having a heterozygous allelic composition of [A/B]. The third group 125 represents those samples having a homozygous-allelic composition of [B/B].

While the above-described example illustrates a sample set which forms three discrete clusters, it will be appreciated that the sample set need not necessarily conform only to this number. Thus, the sample set may include more or less clusters depending on the nature and type of data being analyzed.

For a selected sample set there are typically one or more peripheral or outlier data points 130 whose observed fluorescence properties may not clearly establish with which of the predominant groupings 115, 120, 125 the data point 130 should be associated. Using conventional analytical approaches, the proper allelic composition of these ambiguous or outlier data points 130 may be difficult or impossible to determine with a relatively high degree of certainty or accuracy. Furthermore, when using conventional automated methods for clustering analysis ambiguous data points may be subject to increased miscalling frequencies, flagged for investigator review or omitted from the analysis completely. In various embodiments, the present teachings improve the ability to evaluate and categorize ambiguous data points thereby increasing identification confidence, improving automated sample identification and reducing errors.

FIG. 1B illustrates another exemplary sample set in which fluorescence intensity data is plotted as a log function scatterplot 150. As shown from this graph 150, three distinct groupings 155, 160, 165 corresponding to known homozygous and heterozygous alleles are observable. Ambiguity in data point resolution is further demonstrated by this graph as an overlapping boundary 170 between one of the homozygous groupings 155 and the heterozygous grouping 160. Here each grouping 155, 160, 165 may not be readily resolvable thus impairing visual and automated allelic recognition methods alike. As will be described in greater detail hereinbelow, the present teachings address this potential analytical problem by applying a data classification method which aids in resolution of the data points of the sample set and provides a means for allelic classification and genotyping.

In various embodiments, data grouping may include operations directed towards the development of prototype angles which can be used to characterize and distinguish one cluster from another in a given sample set. As shown in the exemplary scatterplot 173 in FIG. 1C each cluster or allelic grouping may be associated with a discrete angular value 175, 180, 185 based on certain characteristics of the selected cluster. For example, the angular value 175 may be determined for the homozygous cluster [A/A] by evaluating the average or mean of the fluorescence intensity ratios for the data points contained within the cluster and associating the resulting value with a selected origin 190 in the scatterplot 173. Likewise, the angular values 180 and 185 may be determined in a similar manner based on the corresponding heterozygous [A/B] and homozygous [B/B] groupings. As will be described in greater detail hereinbelow angular value determination represents a convenient means by which data points of a sample set may be evaluated with respect to one another and these values may be utilized in the cluster analysis methods as input parameters and subsequently operated upon during the allelic classification operations.

Angular value determination may also be extended to each data point within a selected grouping and the results evaluated to establish appropriate cluster or grouping boundaries. For example, as shown in the exemplary polar plot 191 in FIG. 1D, intensity values 192 for each data point may be plotted as a function of angle values 194 to facilitate cluster analysis. Subsequently, confidence boundaries 196 may be determined based on the methods described herein to aid in associating individual data points with a particular allelic grouping.

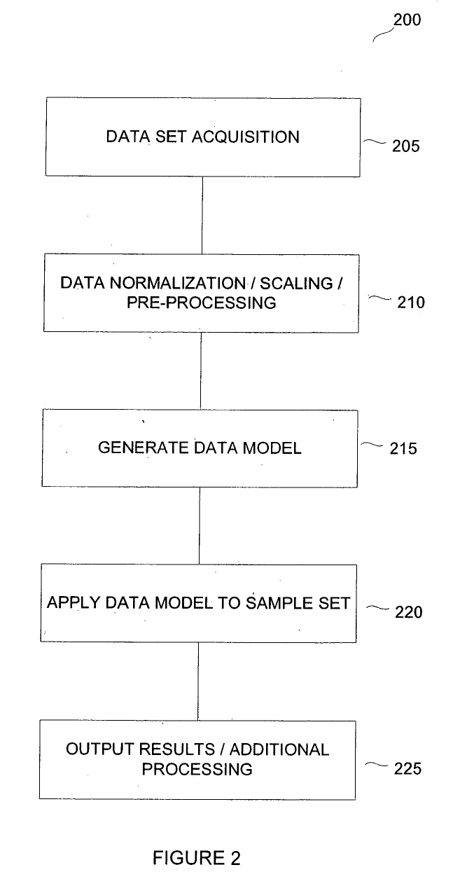

FIG. 2 illustrates a generalized method 200 for SNP analysis according to the present teachings. In one aspect, the method 200 commences in state 205 with the acquisition of sample set information comprising a plurality of data points each having associated component marker or dye intensity values (e.g. red & green fluorescence intensities). The method 200 can operate in conjunction with data acquired from a variety of different sources including, for example, data acquired from dual-label amplification reactions (e.g. Taqman), as well as, array-based detection approaches and other methodologies designed to distinguish alleles on the basis of differences in observable properties including fluorescence, radioactivity, visible light detection, and other approaches. In various embodiments, each data point will possess at least two characteristics or features (e.g. dual-color florescence) which may be used as a basis for discriminating between allelic compositions.

Following data acquisition 205, a normalization, scaling, or pre-processing step 210 may be performed to modify the raw data values of the sample set as desired. This step may involve compensating for background fluorescence, scaling the data to a selected range, adjusting the data to conform to a standardized format, or other such operations to place the data in a form amenable for subsequent processing and analysis.

In one aspect, this step 210 may include a marker or dye correction routine wherein the acquired intensity measurements for a sample or between samples are evaluated. Substantial differences between intensities may indicate that the sample data is not in the same scale and the variations between the intensities may be large enough to affect subsequent clustering analysis. To reduce the potential effect substantial sample intensity differences may have on the analysis, a marker or dye correction factor may be estimated and applied to the data before the clustering analysis is performed.

Additionally, noise correction routines may be applied to the intensity data prior to clustering analysis to improve the quality of the resultant analysis. In one aspect, undesirable noise amplification may be avoided using a detection mechanism wherein the data is first evaluated to determine if a singular cluster exists. In this instance, certain marker or dye corrections may be excluded during the pre-processing step 210 thereby avoiding undesirable increases in noise which might otherwise adversely affect the resulting analysis.

In other embodiments, an origin normalization function may be applied during the pre-processing step 210. In one aspect, the origin normalization function makes use of intensity measurements associated with one or more control samples (e.g. no template controls—NTCs). One purpose of the control samples is to provide a means to determine a background level of fluorescence for each marker or dye. Using this information, the origin normalization function may adjust the intensity values of the data to account for the observed background. In one aspect, data normalization in this manner may be used to adjust the angular measurements of each sample which are dependent on the position of the origin. Additionally, when multiple control samples are present, the origin may be determined by taking the median of the control samples and adjusting the angular values for the data accordingly. Additionally, in instances where control samples are not present or part of the sample set, the origin normalization function may establish a reference origin to allow for determination of the angular measurements for each data point. In one aspect, the normalized origin may be identified by looking for isolated data samples having relatively low fluorescence intensities (e.g. untasked NTCs).

From the aforementioned description it will be appreciated that numerous operations may be performed on the data of the sample set prior to clustering analysis to improve the resultant outcome. It is conceived that various approaches to data processing prior clustering analysis are possible including fluorescence intensity adjustments, changes in sample data representations (e.g. mathematical manipulations including log value determinations and angular value calculations) or other data manipulations desired by the investigator; as such these operations used in conjunction with the below-described clustering analysis approach should be considered to be but other embodiments of the present teachings.

Having suitably adjusted the sample set in state 210, a ML data model is generated in state 215 based on some or all of the resultant data point values. The ML data model is a statistical model which takes a maximum likelihood approach to perform cluster model parameter estimation. Generally, a separate ML data model is developed for each sample set to more accurately reflect the individual and unique characteristics of the selected sample set, however, it will be appreciated that a given ML data model can be applied to one or more sample sets once created. As will be described in greater detail hereinbelow, the ML data model improves on existing clustering approaches by evaluating statistical probabilities from several data point perspectives and combining the results to obtain a model which may be used to more accurately identify the allelic composition for each sample in the sample set.

Once the ML data model has been developed, this model is applied to the data points of the sample set in state 220 to provide a means for determining the appropriate allelic composition for a selected data point. As previously described, one desirable feature of this method 200 is that allelic identification may be performed in a substantially automated manner that it may be adapted to computerized methods and require little or no investigator input or interpretation while still maintaining relatively high degree of allele calling accuracy. Thus, the results of the analysis can be output the investigator in state 225 and other operations such as generating quality values and/or confidence scores can be performed. The resulting information can further be passed to secondary applications for further processing and utilized in subsequent analysis.

In various embodiments, other data types/representations may be used in conjunction with or as a substitute for the aforementioned intensity information. For example, the data used in the allelic identification routines may comprise emission and registration data wherein each signal may be characterized by a peak height and/or peak area. This information can be used in a similar manner as intensity data to develop a likelihood model for purposes of data classification.

Additionally, it is conceived that composite methods may be developed wherein multiple characteristics (e.g. intensity, peak height, and/or peak area) are used in combination with one anther to develop the likelihood model. These characteristics may be further used to develop independent likelihood models which are subsequently evaluated to identify a candidate likelihood model that produces improved results over other potential models. The characteristics used to develop the likelihood models may be correlated or non-correlated to one another and be processed/represented in a number of manners as desired by the investigator.

In various embodiments, the data used in allelic classification may represent consensus-based values wherein the information corresponding to two or more data points may be combined (e.g. duplicate or replicate aggregation). For example, in array-based analytical methods a multiplicity of data points directed towards a similar sample composition may be averaged to generate a consensus value which is then used in allelic classification according to the present teachings. In one aspect, aggregated data may include an associated error estimation and outlier data may be discarded. Likewise other statistical manipulations and data combinations may be conceived for these and other analytical methods to generate input data for allelic classification.

In still further embodiments, the data used in allelic classification may comprise associated uncertainty, variance or tolerance information (e.g. error-bars or quality values). This information may be used in conjunction with the underlying data from which it was obtained and applied in likelihood equation development and evaluation. Additionally, supervised methods may be developed in which training data sets having known compositions are applied to the likelihood model formation methods to aid in generating and ascertaining a suitable likelihood model.

From the foregoing, it will be appreciated that the allelic determination methods of the present teachings may be configured to operate with many different data types and methods of data preparation. Consequently, the below-described use of intensity information as a input data type to the allelic classification methods should be considered as exemplary in nature and not limiting.

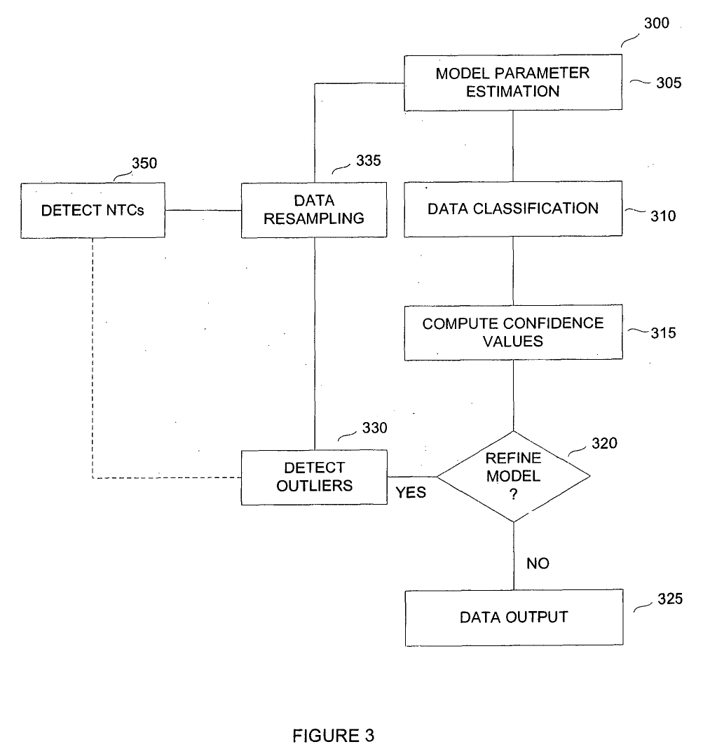

FIG. 3 illustrates a method 300 for data classification which incorporates a maximum likelihood analytical approach as well as model refinement routine to achieve improved allelic identification. As previously described in connection with FIG. 2 above, the input information used by this method 300 may comprise fluorescence data intensities for each data point as well as NTC indices which may be used to identify those data intensities that will be used in background determination and resampling. Additionally, the input data intensities may be normalized or scaled using the NTC information or other approaches.

In state 305, the input data is used in a model parameter estimation function wherein a preliminary model is developed based on the input data as applied to a novel statistical analysis paradigm which takes into consideration various characteristics and assumptions directed towards allelic classification and genotyping. As will be described in greater detail hereinbelow, the data points of the sample set are subjected to a maximum likelihood analysis which may include identifying the number of clusters present in the sample set; determining the mean, variance, or standard deviation of each cluster; and estimating the allele frequency.

In one aspect, the method of allelic classification of the present teachings is distinguished from many conventional methods for clustering analysis based on the manner in which data error or confidence estimates and propagation are handled. Unlike conventional methods which typically track error or confidence estimates and make use of this information downstream of actual allelic classification, the present teachings incorporate an error-weighted clustering approach wherein error or confidence estimates are used in the determination of cluster or data groupings by propagating this information through the classification process.

Another distinguishing feature of the present teachings is the application of an “a priori” identification approach wherein a cluster model is proposed in which various parameters are specified as part of the model and known data values are used to test the model to determine if the resultant values obtained from the model produce an expected result. In one aspect, a suitable likelihood equation which properly associates output of the model with the known data values is taken to be an appropriate equation for subsequent clustering analysis. Considered in another light, the “a priori” model may utilize error information in cluster identification and data classification by testing individual data points against a putative cluster model and evaluating the error information to assess whether or not inclusion of the selected data point in a particular putative cluster generates a statistically valid result.

Based on the aforementioned “a priori” approach, model parameter estimation in state 305 proceeds according to the following rules to generate a putative likelihood function:

(1) Initially, each data cluster in the sample set is considered to be independent of one another with each following a singular distribution. This assessment of the data gives rise to a probability density function p(s) wherein the overall distribution is a mixture distribution defined by the equation:

$$Equation 1 \text{:} p ( s ) = P ( {C}_{i} ) {\sum }_{i} {p}_{i} ( s )$$

In this equation P(Cᵢ) represents the “a priori” probabilities of each cluster and pᵢ(s) represents the probability density function for a cluster Cᵢ with s denoting a selected sample data point.

(2) In allelic classification it is generally observed that each of the clusters tend to follow a binomial distribution (e.g. Hardy-Weinberg equilibrium) wherein a relatively large population is assumed insuring minimal sampling error with independent allelic frequencies. Supposing that the allele frequency for a first allele “A” is “p” and the allele frequency for a second allele “B” is “q” then it generally holds that: (p+q)=1 (e.g. probability sum=1) and 1−q=p.

Consequently, the allelic frequencies related to the distribution of three clusters (2 homozygous [A/A] and [B/B] and one heterozygous [A/B]) may be defined by the equation:

p²(AA)+2pq(AB)+q²(BB)=1 2

This equation may be generated based on the observation that for the two alleles, the distribution of possibilities are equal to the square of the allele possibilities or

(p(A)+q(B))² =p²(AA)+2pq(AB)+q²(BB)

Alternatively the probability of generating a specific allele which is equal to the allele frequency can be diagrammed as shown in Table 1 by the exemplary Punnett square which can be summed to p²(AA)+2pq(AB)+q²(BB).

TABLE 1

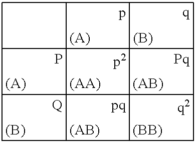

(3) In calculating the angle for the data points in each cluster, a conditional Gaussian distribution is followed according the equation:

$$Equation 3 \text{:} {p}_{i} ( θ \vert r ) = \frac{1}{\sqrt{2 π} {σ}_{i , r}} exp \{ \frac{{( θ - {\overline{θ}}_{i} )}^{2}}{2 {σ}_{i , r}^{2}} \}$$

In this equation, \overline{θ}ᵢ represents the mean angle of a cluster Cᵢ with σ_{i,r} representing a parameter inversely proportional to the observed intensity r.

(4) In various sample sets it is observed that there may be outlier data points which tend not to clearly fall into one of the identified clusters or data groupings. In one aspect, the allelic classification and genotyping according to the present teachings provide for a knowledge-based means for outlier detection.

Based on the aforementioned principals, for a selected sample set, the maximum likelihood (ML) criteria is used to estimate the model parameters with the likelihood function defined as the joint probability density function of the data points in the sample set. This likelihood function can be represented as:

L=1n p{x₁,Λ,xₙ} 4

In this equation xₙ, n=1,Λ N denotes all N samples which if all samples are considered independent results in the following likelihood function:

$$Equation 5 \text{:} \begin{matrix}L = {\sum }_{n = 1}^{N} ln p \{ {x}_{n} \} \\ = {\sum }_{n = 1}^{N} ln {\sum }_{j} P \{ {x}_{n} , {C}_{j} \} \\ = {\sum }_{n = 1}^{N} ln {\sum }_{j} p \{ x \vert {C}_{j} \} P \{ {C}_{j} \}\end{matrix}$$

The maximum likelihood estimation of parameters in state 305 can thus be obtained by maximizing the above-indicated likelihood function.

Referring again to FIG. 3, having identified a suitable parameter set in state 305, the method 300 proceeds to a state 310 wherein data classification takes place based on the statistical model provided by the likelihood function. In one aspect, a Bayes classifier approach is employed to perform the allele-calling operation (e.g. associating a selected data point with one of the homozygous or heterozygous clusters). Briefly described, this classifier approach makes use of a posteriori probability analysis which establishes a data model and determines the probability that each selected data point belongs to the cluster based on a probability model. In general this approach applies an inverse conditional logic to make predictions as to which cluster a selected data point belongs (maximum posteriori probability) and may be modeled by a following rule-based decision equation the use of which will be described in greater detail hereinbelow:

xεCⱼ, where j=argᵢ max P(Cᵢ |x) 6

Following data classification in state 310, the method 300 proceeds to state 315 wherein confidence values are assessed for each data point in the sample set. In various embodiments, the statistical framework for which confidence values are determined is based upon the combination of several assumed statistical probabilities (e.g. a probability function based on individual data point probabilities). This manner of confidence value determination is distinguished from conventional methods which rely on training data sets, data models, and neural network approaches to achieve a relatively high quality estimation of the allele call confidence for each data point. During this state 315, additional computations may also be performed including establishing probable outliers and calculating overall sample scores for a selected sample set (e.g. plate or array score).

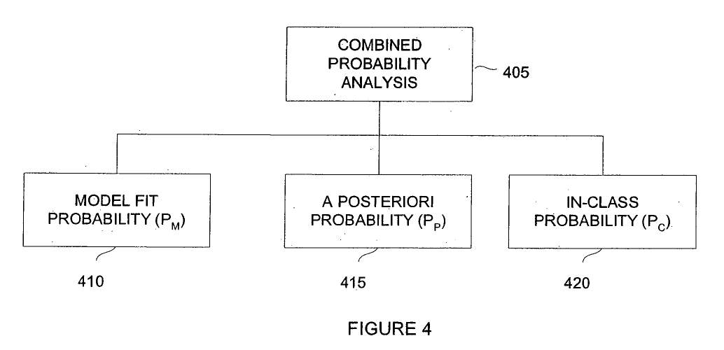

In general, confidence value determination according to the present teachings follows a joint probability analysis wherein statistical assessments are performed as a function of various experimental and analytical parameters which are subsequently combined to generate a confidence value for each data point. For example, in allelic classification, confidence value determination may include combined statistical analysis at the level of: (a) the likelihood function or model itself, (b) the data cluster and (c) the sample data. Additional details of the confidence value determination will be described in conjunction with FIG. 4 below.

In various embodiments, the aforementioned steps represent a first pass analysis of the data points of the sample set and provide an initial foundation of information which helps label and determine the structure or arrangement of the data points relative to one another. Furthermore, the first pass analysis aids in detecting outlier data points which can be identified for the purposes of reformulating the model in subsequent passes.

Having performed the preliminary or “first pass” data classification, the method 300 reaches a branch state 320 where the data may be output in state 325 or alternatively, additional refinement of the model may take place. In various embodiments, one or more “refinement passes” may be made to refine the model used to classify the data. Generally, as few as a single refinement pass significantly improves the model characteristics to increase the overall accuracy of allelic classification for the sample set.

Model refinement may proceed in state 330 wherein “outlier data” is detected. Outlier data reflects those data points which do not generally fall within the bounds of a single cluster and therefore may be difficult to classify. The determination of what constitutes outlier data is flexibly defined and may for example be based on statistical analysis of the intensity or angular values for each data point. Data points which exceed a threshold value, defined for example by the mean value for a cluster, may be excluded from the analysis and subsequently the remaining data points may be used to define a resampling set in state 335.

The resampling set may then be used as input in state 305 to perform a subsequent round of model parameter estimations and the data classified and confidence values computed as described above. One desirable feature of the present teachings is the ability to provide increased classification accuracy through model refinement without additional training data using the existing data points of the sample set.

In various embodiments, for example in array-based allelic analysis, model refinement may further comprise detecting or identifying NTCs which may be present (state 350). Information associated with NTCs such as those not previously utilized in data normalization or scaling as described above may be used in resampling in state 335. For example, NTCs may be used to define a new origin from which angular measurements for each data point and cluster are made to improve the quality of classification.

Following the second (or third, fourth, etc.) pass data analysis, the output genotypes and quality values may be distributed in state 325. In various embodiments, the output data may be saved to a database or other storage means, presented to the user for inspection, or the redirected to another application or instrument for additional post-processing. For example, data output may be subjected to a filtering routine which identifies low quality data points, bad samples, or erroneous runs. These and other post-processing routines used in conjunction with the aforementioned analytical methods should be considered to be but other embodiments of the present teachings.

As will be appreciated by one of skill in the art, the number of iterations used to refine the likelihood equation and perform allelic classification is not necessarily rigid. In certain circumstances, a single pass data analysis may be sufficient to generate a likelihood equation of good predicative quality. In other instances, likelihood equation development may desirably occur over multiple iterations of the aforementioned steps. Furthermore, it will be appreciated that the order of the steps may be altered as desired without deviating from the scope of the present teachings. For example, the determination for model refinement 320 may precede confidence value determination 315. Additionally, other steps may be included in the method 300, for example, data processing steps including sample data integration or consensus determination may occur following data resampling 335. Consequently, these and other modifications to the method for allelic determination are considered but other embodiments of the present teachings.

In various embodiments, the data resampling step 335 may be used to reduce or increase the number of data points in the sample set. For example, in addition to discarding outlier data, data resampling may generate additional data points on the basis of the input sample information passed through the first iteration of the likelihood equation determination. This approach may be weighted on the basis of error, uncertainty, or other information to skew, direct, or favor the development of a particular type or quality of likelihood equation.

In one aspect, error determination approaches may be incorporated into the allelic determination methods wherein each allele call may be associated with a corresponding error or uncertainty value. The uncertainty value may further be determined by error propagation methods wherein the uncertainty in the allele call is monitored over one or more iterations of the likelihood equation determination. This error information may correspond to error information propagated through the theoretical error modeling process (e.g. shot noise) and model fits (e.g. chi squared) to the empirical cluster model used in likelihood calculation.

FIG. 4 illustrates the probability components of a combined statistical analysis 405 for data point evaluation. The model comprises three probability components P_{M}410, Pₚ415, and P_{c}420 wherein P_{M}410 represents a model fit probability analysis, Pₚ415 represents a posterior probability analysis for a selected cluster, and P_{C}420 represents a cluster fit probability analysis for a selected data point. The model fit probability P_{M}410 may be used to estimate the confidence of the likelihood model itself and in general measures how well sample points may fit into the model; the posteriori probability Pₚ415 may be used to estimate the probability that a selected data point belongs to the assigned allelic or genotype cluster C given the estimated model; and the in-class probability P_{c}420 may be used to estimate the probability that a selected cluster could produce a particular data point given a cluster in a particular model.

The product of these probabilities may be then taken to yield a composite probability that a data point “s” has the assigned genotype generated by a selected system (e.g. a joint probability that described the correctness of the genotyping decision). An equation representing the composite probability is given by:

P{S,SεC,M}=P_{M}·Pₚ{SεC|M}·P_{c}{s |M,sεC} 8

Using the estimated model as a basis, the posteriori probability Pₚ 415 can be calculated with a relatively high degree of accuracy with the model fit probability P_{M} 410 and in-class probability P_{C} 420 being subjectively estimated based, in part, on the definition of the model fit. Additionally, it is noted that the perceived confidence value is generally related to the probability of decision (which are not necessarily the same) and as a consequence the perceived confidences may be determined as an empirical function of the probability of decision. Taken together, the composite function of probabilities forms a confidence value cv described by the equation:

cv=f(P{s,sεC,M})=f(P_{M},Pₚ,P_{c})=f₁(P_{M})·f₂(Pₚ)·f₃(P_{c}) 9

Details of each of the component probabilities 410, 415, 420 and their application in the combined analysis 405 will be described in greater detail hereinbelow.

A posteriori probability Pₚ

The a posteriori probability calculation generally attempts to establish what the probability is for a selected data point to fit within a selected cluster relative to other clusters. As previously noted, a posteriori probability indicates the likelihood of a selected data point “x” belonging to a particular cluster based on the estimated statistical model reflected by the conditional Cⱼ. When the statistical model is estimated, the a posteriori probability may be calculated using a Bayes approach. For additional details of how a posteriori probability may be applied in Bayes decision theory the reader is referred to: Duda, R. and Hart, P.; “Pattern Classification and Scene Analysis”; John Wiley; New York; 1973. In one aspect, the a posteriori probability may be determined according to the following equations:

$$\begin{matrix}Equation 10 \text{:} P ( {C}_{j} \vert x ) = \frac{p ( x \vert {C}_{j} ) \cdot P ( {C}_{j} )}{p ( x )} \\ Equation 11 \text{:} p ( x ) = {\sum }_{j = 1}^{n} p ( x \vert {C}_{j} ) \cdot P ( {C}_{j} )\end{matrix}$$

In these equations, the a priori probability P(Cᵢ) can be derived from the allele frequencies by assuming the major allele frequency is p and the minor allele frequency is q=1−p. From this, the a priori probabilities can be determined as:

P(C₁)=p² 11

P(C₂)=2pq 12

P(C₃)=q²

According to these equations P(C₁) reflects the probability of having a major homozygous SNP (e.g. [A/A]), P(C₂) reflects the probability of having a heterozygous SNP (e.g. [A/B]), and P(C₃) reflects the probability of having a minor homozygous SNP (e.g. [B/B]).

Model fit probability P_{M}

In one aspect, data point analysis can be considered from the perspective of model fit, the application of which generally affects every data point. This probability attempts to estimate how good the fit is between the data points and the model. The model fit probability may be determined using the likelihood function as a measurement of model fit and defined by the equation:

$$Equation 14 \text{:} \begin{matrix}L = {\sum }_{n = 1}^{N} ln p \{ {x}_{n} \} \\ = {\sum }_{n = 1}^{N} ln {\sum }_{j} P \{ {x}_{n} , {C}_{j} \} \\ = {\sum }_{n = 1}^{N} ln {\sum }_{j} p \{ x \vert {C}_{j} \} P \{ {C}_{j} \}\end{matrix}$$

In this equation Xₙ,n=1, . . . ,N are representative of data points within the sample set. Observing that the distribution of the posteriori probability itself may be able to provide information about the model fit, the model fit probability may be defined as a function of the likelihood function and the distribution of he posteriori probabilities or all data points which can be calculated according to the equation:

P_{M}=ƒ(L,pₚ) 15

In-class probability P_{c}

In general, the “in-class probability” may reflect the probability that a given data point is generated by the assigned genotype class given the estimated model. This probability analysis considers the position or location of a selected data point within a cluster (e.g. middle of cluster vs. boundary). This probability may be estimated from both the angle difference between the point and the model angle mean and the intensity difference between the data point and the model mean intensity. In one aspect, the probability estimate is computed form a separable two dimensional Gaussian function in the polar domain (e.g. the angle-intensity domain) defined by the equation:

$$Equation 16 \text{:} {P}_{c} ( r , θ ) = exp ( \frac{{\vert r - {r}_{m} \vert}^{2}}{k \cdot 2 {σ}_{r}^{2}} ) \cdot exp ( \frac{{\vert θ - {θ}_{m} \vert}^{2}}{k \cdot 2 {σ}_{θ}^{2}} )$$

In the equation r reflects the data point intensity with rₘ reflecting the mean model intensity, θ reflects a sample point angle with θₘ reflecting the mean model angle, σᵣ and σ_{θ} reflect the standard deviations for the intensity and angle respectively, and k is a scaling factor used to scale of the confidence value.

According to this equation, a first Gaussian function may be used to represent the distribution of angles in the cluster with a second Gaussian function used to represent the distribution of intensities. Additionally, the mean and the standard deviations for the intensities and the angles may be calculated form the data points assigned to the clusters.

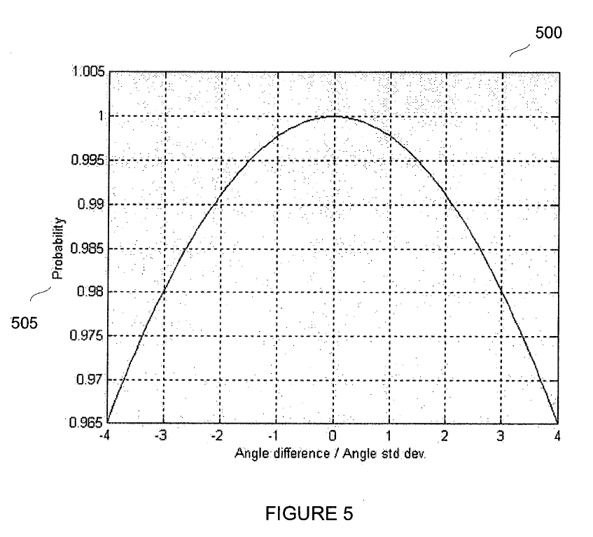

FIG. 5 illustrates an exemplary Gaussian function 500 shown in angle space wherein the parameters for this function are estimated from the data points assigned to the cluster. As previously noted, the measured standard deviation of the angles may be scaled by a selected factor in order to calibrate the resulting probability estimates 505. For example, a scale factor k may be set so that an angle difference of 4σ_{θ} results in a probability (P-value) of approximately 96.5%. Scaling in this manner may be used to include data points that are within 4σ_{θ} from the mean in the associated cluster when the confidence value threshold is set at approximately 95%. It will be appreciated that such scaling can be done for a variety of different values to achieve different degrees of selectivity and sensitivity during the data analysis. A similar Gaussian function and scaling means may also be applies to the intensity values for the data points of the sample set (not shown).

From the foregoing it will be appreciated that the methods described herein provide a means for allele calling and genotyping using a statistical model based clustering approach combined with knowledge from specific applications. These methods provide a unified framework for allele-calling in many different contexts and may be applied to the data acquired from various identification methodologies including, for example: Taqman-based approaches, array-based identification schemes, as well as capillary electrophoresis data (e.g. SMPlex data). Additionally, various error propagation methods used to generate error estimates and confidence values from the various aforementioned identification methodologies may be used an input to clustering methods prior to analysis and allele calling. Furthermore, while the principles and structure of the methods remain generally similar for different applications, various method parameters and thresholds may be adjusted according to the specific characteristics of the data used in the application thus improving the flexibility of the methods to be used in other contexts.

In addition to the analytical means described above for likelihood model development, other model fitting methods may be used in place of or in connection with the allelic clustering approach. For example, chi-square fitting approaches, K-means clustering, machine learning approaches, and neural networks may be used to develop a suitable likelihood equation for data evaluation and allelic determination. Furthermore, clustering confidence can be assessed using a selected likelihood model and a known sample set to assess the probability that the identified cluster characteristics (e.g. center/boundaries) are acceptable. One function of this “sanity check” is to assess whether or not a selected likelihood function associates a selected data point with the proper or expected cluster and associated allele call.

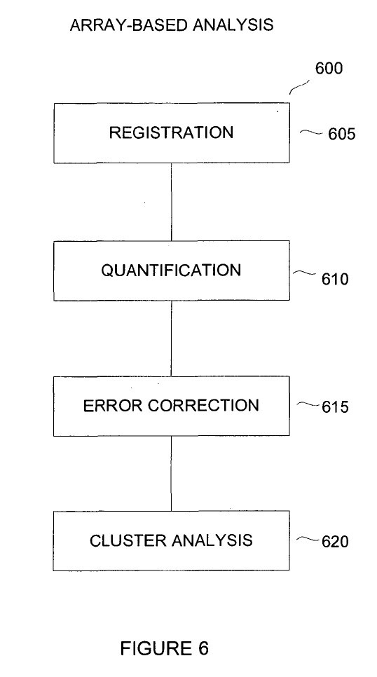

FIG. 6 illustrates an exemplary method 600 for array-based analysis applying the allele classification approach of the present teachings. In various embodiments, this method 600 commences in state 605 with a signal registration and sample identification operation. In general, signals associated with an array have a known location which can be associated with a particular sample composition. Thus for an array used in SNP analysis signals arising from different positions on the array may each be associated with a corresponding SNP component. In one aspect, a decode file or signal/sample identification mask may be used to make the proper associations to be used in analyzing the array.

Subsequently, in state 610 the signals associated with particular positions on the array may be quantified. In certain embodiments, replicates may be aggregated and error estimates may be performed with aggregate errors propagated for further analysis.

In state 615, error correction routines may be employed which may include the analysis of control signal information, expected distribution fits, normalizations, and other operations designed to prepare the array data for further processing.

Taken together, in state 620, the aforementioned information may then be used as input and used in conjunction with the allelic classification methods previously described and subsequently presented to the investigator or made ready for post-processing by other applications or instruments.

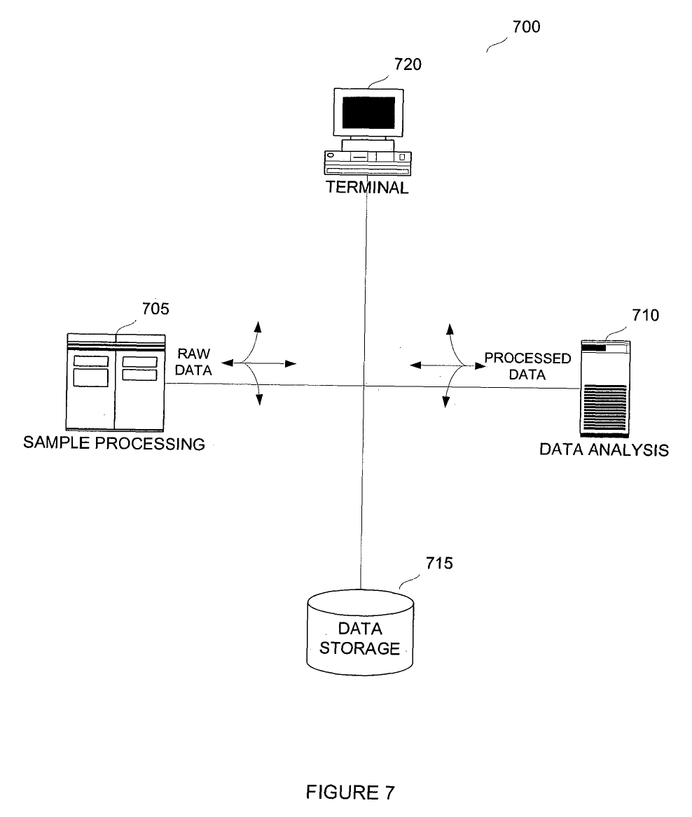

FIG. 7 illustrates an exemplary system 700 which may be used to perform allelic classification according to the aforementioned methods. In one aspect, a sample processing component 705 may provide means for performing operations associated with sample processing and data acquisition. These operations may include by way of example; labeling, amplifying, and/or reacting the sample in the presence of a suitable marker or label; exposing the sample to an appropriate analysis substrate or medium; and detecting signals or emissions from the sample which will serve as input data for the allelic classification methods. Instruments which may be associated with these operations include but are not limited to array-analysis instruments, sequencing instruments, fluorescent signal detection instruments, thermalcyclers, and other such instruments used in sample processing and data acquisition.

Raw data provided by the sample processing component 705 may be subsequently stored in a data storage component 715. This component 715 may comprise any of various types of devices designed for storing of data and information including for example; hard disk drives, tape drives, optical storage media, random access memory, read-only memory, programmable flash memory devices and other computers or electronic components. Furthermore, the data and information obtained from the sample processing component 705 may be stored and organized in a database, spreadsheet, or other suitable data structure, data storage object, or application which operates in connection with the data storage component 715.

In various embodiments, a data analysis component 710 may be present within the system 700. This component 710 possesses functionality for acquiring data and information from the sample processing component 705 or the data storage component 715. The data analysis component 710 may further provide a hardware or software implementation of the aforementioned allelic classification methods. In one aspect, the data analysis component 710 is configured to receive input data and may return processed data including allelic classifications or genotyping information which may be stored in the data storage component 715 or displayed directly to the investigator via a display terminal 720.

Each of the functionalities of the aforementioned components 705, 710, 715, 720 may be integrated into a singular hardware device or into one or more discrete devices. These devices may further possess network connectivity facilitating communications and data transfer between the devices as desired by the investigator. It will be appreciated that numerous suitable hardware and software configurations may be developed which implement the allelic classification methods of the present teachings, as such each of these configurations should be considered but other embodiments of the present teachings.

Although the above-disclosed embodiments of the present invention have shown, described, and pointed out the fundamental novel features of the invention as applied to the above-disclosed embodiments, it should be understood that various omissions, substitutions, and changes in the form of the detail of the devices, systems, and/or methods illustrated may be made by those skilled in the art without departing from the scope of the present invention. Consequently, the scope of the invention should not be limited to the foregoing description, but should be defined by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. A method for allelic classification, the method comprising:
acquiring intensity information for a plurality of samples wherein the intensity information comprises a first intensity component associated with a first allele and a second intensity component associated with a second allele;
evaluating the intensity information for each of the plurality of samples to identify one or more data clusters, each cluster associated with a discrete allelic combination and determined, in part, by comparing the first intensity component relative to the second intensity component;
generating a likelihood model that predicts the probability that a selected sample will reside within a particular data cluster based upon its intensity information; and
applying the likelihood model to each of the plurality of samples to determine its associated allelic composition.

2. The method of claim 1, wherein the likelihood model comprises a model-fit probability assessment that estimates confidence in the likelihood model itself and assesses how well a selected sample and its respective intensity information fit the model.

3. The method of claim 1, wherein the likelihood model comprises an in-class probability assessment that estimates the probability that a selected cluster identifies a selected sample and its respective intensity information.

4. The method of claim 1, wherein the likelihood model comprises an a posteriori probability assessment that estimates the probability of a selected sample and its respective intensity information belonging to an assigned cluster.

5. The method of claim 1, wherein the data clusters comprise at least three discrete clusters each associated with a different allelic classification.

6. The method of claim 5, wherein the data clusters comprise a first cluster type associated with a first homozygous allelic classification.

7. The method of claim 6, wherein the data clusters comprise a second cluster type associated with a first heterozygous allelic classification.

8. The method of claim 7, wherein the data clusters comprise a third cluster type associated with a second homozygous allelic classification.

9. The method of claim 1, wherein the allelic classification is used to perform a mutational analysis of one or more samples.

10. The method of claim 1, wherein the allelic classification is used to perform a single nucleotide polymorphism analysis of one or more samples.

11. The method of claim 1, wherein the geneotype for one or more samples is identified by performing the allelic classification.

12. The method of claim 1, wherein the intensity information for the plurality of clusters is normalized.

13. The method of claim 1, wherein the plurality of samples comprise at least one “no template control” sample and associated intensity information that is used for the purposes of sample scaling.

14. The method of claim 1, wherein the likelihood model is generated in an iterative manner to refine the likelihood model.

15. The method of claim 14, wherein two or more iterations are used to generate a refined likelihood model.

16. The method of claim 14, wherein refinement of the likelihood model is performed by identifying outlier samples and removing these samples prior to further likelihood model generation to generate a refined likelihood model.

17. The method of claim 14, wherein refinement of the likelihood model comprises performing a data resampling operation wherein a subset of the plurality of samples are used to generate the refined likelihood model.

18. The method of claim 1, wherein the first and second intensity components of the intensity information comprise fluorescence intensities associated with discrete markers or labels.

19. The method of claim 1, wherein the intensity information for each sample is acquired from a dual-label amplification protocol.

20. The method of claim 19, wherein the dual-label amplification protocol comprises a Taqman or SNPlex protocol.

21. The method of claim 1, wherein the intensity information for each sample is acquired from an array-based detection protocol.

22. A method for clustering analysis, the method comprising:
identifying a sample set comprising a plurality of data points, each data point having an angular value representative of an association between a first and a second intensity component;
generating a likelihood model and associated parameter set wherein the angular values of the data points are used in determining the appropriate parameters to be used in the likelihood model and wherein the efficacy of the likelihood model is assessed by evaluating the probability the likelihood model properly identifies selected data points in the sample set;
applying the likelihood model to the plurality of data points within the sample set and grouping the data points into discrete clusters; and
associating a selected classification with each discrete cluster and its component data points.

23. The method of claim 22, wherein the clustering analysis is used in allelic classification.

24. The method of claim 23, wherein the allelic classification comprises identifying the discrete clusters representing a homozygous allelic classification or a heterozygous allelic classification and associating the data points of a particular cluster with the identified allelic classification.

25. The method of claim 23, wherein at least three discrete clusters exist which correspond to a first homozygous allelic classification, a second homozygous allelic classification, and a first heterozygous allelic classification.

26. The method of claim 22, wherein the clustering analysis is used to perform mutational analysis.

27. The method of claim 22, wherein the clustering analysis is used to perform single nucleotide polymorphism analysis.

28. The method of claim 22, wherein the likelihood model and associated parameter set are evaluated using a probability assessment the estimates confidence in the likelihood model itself and assesses how well as selected data point fits the model using the associated parameter set.

29. The method of claim 22, wherein the likelihood model and associated parameter set are evaluated using a probability assessment that estimates the probability that a selected cluster properly identifies a selected data point associated with the cluster.

30. The method of claim 22, wherein the likelihood model and associated parameter set are evaluated using a probability assessment that estimates the probability that a selected data point belongs to the cluster to which it is grouped.

31. The method of claim 22, wherein the likelihood model and associated parameter set are generated in an iterative manner wherein one or more data points are excluded from the model and parameter analysis and a second refined model and parameter set is generated using the remaining data points.

32. The method of claim 31, wherein the excluded data points comprise outlier data points which reside beyond a defined cluster threshold.

33. The method of claim 31, wherein additional refinements to the model and parameter set are performed by excluding additional data points.

34. A method for allelic classification, the method comprising:
identifying a sample set comprising a plurality of data points each having at least two component intensity values;
evaluating the component intensity values for the plurality of data points to group the data points into one or more data clusters representative of discrete allelic classifications;
generating a likelihood function that describes the grouping of a selected data point using its component intensity value; and
associating an allelic classification with each data point using the likelihood function.

35. The method of claim 34, further comprising performing a confidence value assessment for each data point indicative of a degree of confidence with which the allelic classification is made.

36. The method of claim 34, further comprising a refinement operation in which at least one data point is excluded from the sample set and a refined likelihood function is generated based on the remaining data points of the sample set.

37. The method of claim 36, wherein the at least one excluded data point comprises outlier data which resides outside of a selected grouping.

38. The method of claim 34, wherein at least three groupings of data points are present and correspond to a first homozygous allelic classification, a second allelic classification and a first heterozygous classification.

39. The method of claim 34, wherein the likelihood function efficacy is further evaluated based on the confidence of the likelihood model itself and how well data points fit into the model.

40. The method of claim 34, wherein the likelihood function efficacy is further evaluated according to the probability that a selected data point belongs to the associated allelic classification.

41. The method of claim 34, wherein the likelihood function efficacy is further evaluated according to the probability that a selected data cluster could be associated with a particular data point.

42. A computer readable medium having stored thereon instructions which cause a general purpose computer to perform the steps of:
acquiring experimental information for a plurality of samples wherein the experimental information comprises a first data component associated with a first allele and a second data component associated with a second allele;
evaluating the experimental information for each of the plurality of samples to identify one or more data clusters, each cluster associated with a discrete allelic combination and determined, in part, by comparing the first data component relative to the second data component;
generating a likelihood model that predicts the probability that a selected sample will reside within a particular data cluster based upon its experimental information; and
applying the likelihood model to each of the plurality of samples to determine its associated allelic composition.

43. The computer readable medium of claim 42, wherein the first and second data component comprise sample intensity information.

44. The computer readable medium of claim 43, wherein the sample intensity information is acquired following reacting each sample using a dual-label amplification protocol.

45. The computer readable medium of claim 44, wherein the dual-label amplification protocol comprises a Taqman or SNPlex protocol.

46. The computer readable medium of claim 42, wherein the likelihood model comprises a model-fit probability assessment that estimates confidence in the likelihood model itself and assesses how well a selected sample and its respective experimental information fit the model.

47. The computer readable medium of claim 42, wherein the likelihood model comprises an in-class probability assessment that estimates the probability that a selected cluster identifies a selected sample and its respective experimental information.

48. The computer readable medium of claim 42, wherein the likelihood model comprises an a posteriori probability assessment that estimates the probability of a selected sample and its respective experimental information belonging to an assigned cluster.

49. The computer readable medium of claim 42, wherein the data clusters comprise at least three discrete clusters each associated with a different allelic classification.

50. The computer readable medium of claim 49, wherein the data clusters comprise a first cluster type associated with a first homozygous allelic classification, a second cluster type associated with a first heterozygous allelic classification, and a third cluster type associated with a second homozygous allelic classification.

51. The computer readable medium of claim 42, wherein the data clusters comprise one or more clusters each associated with a discrete allelic classification.

52. The computer readable medium of claim 42, wherein the steps further comprise normalizing the experimental information.

53. The computer readable medium of claim 42, wherein the steps further operate in an iterative manner to refine the likelihood model.

54. The computer readable medium of claim 53, wherein two of more iterations are used to generate a refined likelihood model.

55. The computer readable medium of claim 54, wherein the likelihood model is refined by identifying outlier samples and removing these samples prior to further likelihood model generation.

56. The computer readable medium of claim 42, wherein the experimental information comprises angular data.

57. The computer readable medium of claim 56, wherein the angular data is generated by comparing the first data component with the second data component for each sample.

58. The computer readable medium of claim 56, wherein the angular data reflects a ratio between the first data component and the second data component for each sample.

59. A computer readable medium having stored thereon instructions which cause a general purpose computer to perform the steps of:
identifying a sample set comprising a plurality of data points, each data point having an angular value representative of an association between a first and a second intensity component;
generating a likelihood model and associated parameter set wherein the angular values of the data points are used in determining the appropriate parameters to be used in the likelihood model and wherein the efficacy of the likelihood model is assessed by evaluating the probability the likelihood model properly identifies selected data points in the sample set;
applying the likelihood model to the plurality of data points within the sample set and grouping the data points into discrete clusters; and
associating a selected classification with each discrete cluster and its component data points.

60. The computer readable medium of claim 59, wherein the operations are used to perform allelic classification in which the discrete clusters represent a homozygous allelic classification or a heterozygous allelic classification and the data points of a particular cluster are associated with the corresponding allelic classification.

61. The computer readable medium of claim 60, wherein at least three discrete clusters exist which correspond to a first homozygous allelic classification, a second homozygous allelic classification, and a first heterozygous allelic classification.

62. The computer readable medium of claim 59, wherein the likelihood model and associated parameter set are evaluated using a probability assessment the estimates confidence in the likelihood model itself and assesses how well as selected data point fits the model using the associated parameter set.

63. The computer readable medium of claim 59, wherein the likelihood model and associated parameter set are evaluated using a probability assessment that estimates the probability that a selected cluster properly identifies a selected data point associated with the cluster.

64. The computer readable medium of claim 59, wherein the likelihood model and associated parameter set are evaluated using a probability assessment that estimates the probability that a selected data point belongs to the cluster to which it is grouped.

65. The computer readable medium of claim 59, wherein the likelihood model and associated parameter set are generated in an iterative manner wherein one or more data points are excluded from the model and parameter analysis and a second refined model and parameter set is generated using the remaining data points.

66. A computer readable medium having stored thereon instructions which cause a general purpose computer to perform the steps of:
identifying a sample set comprising a plurality of data points each having at least two component experimental values;
evaluating the component experimental values for the plurality of data points to group the data points into one or more data clusters representative of discrete allelic classifications;
generating a likelihood function that describes the grouping of a selected data point using its component experimental value; and
associating an allelic classification with each data point using the likelihood function.

67. The computer readable medium of claim 66, the steps further comprising performing a confidence value assessment for each data point indicative of a degree of confidence with which the allelic classification is made.

68. The computer readable medium of claim 66, the steps further comprising a refinement operation in which at least one data point is excluded from the sample set and a refined likelihood function is generated based on the remaining data points of the sample set.

69. A computer-based system for performing allelic classification, the system comprising:
a database for storing experimental information for a plurality of samples, the experimental information reflecting the allelic composition of each sample;
a program which performs the operations of:
retrieving experimental information for the plurality of samples from the database wherein the experimental information comprises a first data component associated with a first allele and a second data component associated with a second allele;
evaluating the experimental information for each of the plurality of samples to identify one or more data clusters, each cluster associated with a discrete allelic combination and determined, in part, by comparing the first experimental component relative to the experimental component;
generating a likelihood model comprising a model-fit probability assessment that estimates confidence in the likelihood model itself and assesses how well a selected sample and its respective experimental information fit the model, the model further used to predict the probability that a selected sample is associated with a particular data cluster based upon its experimental information; and
applying the likelihood model to each of the plurality of samples to determine its associated allelic composition.

70. The system of claim 69, wherein the first and second data component comprise sample intensity information.

71. The system of claim 69, wherein the likelihood model comprises an in-class probability assessment that estimates the probability that a selected cluster identifies a selected sample and its respective experimental information.

72. The system of claim 69, wherein the likelihood model comprises an a posteriori probability assessment that estimates the probability of a selected sample and its respective experimental information belonging to an assigned cluster.

73. The system of claim 69, wherein the data clusters comprise at least three discrete clusters each associated with a different allelic classification.

74. The system of claim 73, wherein the data clusters comprise a first cluster type associated with a first homozygous allelic classification, a second cluster type associated with a first heterozygous allelic classification, and a third cluster type associated with a second homozygous allelic classification.

75. The system of claim 69, wherein the program further operates to normalize the experimental information.

76. The system of claim 69, wherein the program further operates in an iterative manner to refine the likelihood model.

77. The system of claim 76, wherein two of more iterations are used to generate a refined likelihood model.

78. The system of claim 76, wherein the program refines the likelihood model by identifying outlier samples and removing these samples prior to further likelihood model generation to generate the refined likelihood model.

79. The system of claim 69, wherein the experimental information comprises angular data generated by comparing the first data component with the second data component for each sample.

80. A computer-based system for performing allelic classification, the system comprising:
a database for storing experimental information for a plurality of samples, the experimental information reflecting the allelic composition of each sample; and
a program which performs the operations of:
identifying a sample set comprising a plurality of data points, each data point having an angular value representative of an association between a first and a second intensity component;
generating a likelihood model and associated parameter set wherein the angular values of the data points are used in determining the appropriate parameters to be used in the likelihood model and wherein the efficacy of the likelihood model is assessed by evaluating the probability the likelihood model properly identifies selected data points in the sample set;
applying the likelihood model to the plurality of data points within the sample set and grouping the data points into discrete clusters; and
associating a selected classification with each discrete cluster and its component data points.

81. The system of claim 80, wherein the clustering analysis is used in allelic classification by identifying the discrete clusters representing a homozygous allelic classification or a heterozygous allelic classification and associating the data points of a particular cluster with the identified allelic classification.

82. The system of claim 81, wherein at least three discrete clusters exist which correspond to a first homozygous allelic classification, a second homozygous allelic classification, and a first heterozygous allelic classification.

83. The system of claim 80, wherein the likelihood model and associated parameter set are evaluated using a probability assessment the estimates confidence in the likelihood model itself and assesses how well as selected data point fits the model using the associated parameter set.
